(19) United States
(12) Patent Application Publication
(10) Pub. No.: US 2026/0022328 A1
(43) Pub. Date: Jan. 22, 2026

(54) CONTROLLING CONTAMINANTS DURING FERMENTATION

(71) Applicant: NewLeaf Symbiotics, Inc., St. Louis, MO (US)

(72) Inventors: Aaron Kelley, Chesterfield, MO (US); Anthony NEUMANN,, St. Louis, MO (US)

(21) Appl. No.: 18/997,218

(22) PCT Filed: Jul. 21, 2023
(86) PCT No.: PCT/US2023/070665

§ 371 Date: Jan. 21, 2025

Related U.S. Application Data
(60) Provisional application No. 63/391,007, filed on Jul. 21, 2022.

(51) Int. Cl. C12N 1/20; A01N 35/06; A01N 65/08; A01P 1/00

(52) U.S. Cl. CPC C12N 1/205; A01N 35/06; A01N 65/08; A01P 1/00

(57) ABSTRACT

Methods and compositions for use in growth of pink-pigmented facultative methylotrophs (PPFM) and/or methanotroph bacteria are provided herein, wherein the fermentation comprises hop acids to inhibit growth of bacterial contaminants.

### CROSS REFERENCE TO RELATED APPLICATIONS

This patent application claims the benefit of U.S. Provisional Patent Application No. 63/391,007, filed Jul. 21, 2022; the entire disclosure of which are incorporated herein by reference.

### FIELD OF THE INVENTION

The invention relates to the fields of microbial cultivation. More specifically, methods are provided for controlling contaminants during commercial fermentation of pink pigmented facultative methylotrophs (PPFM), such as species of Methylobacterium and Methylorubrum, and during fermentation of methanotroph bacteria, including species of Methylomicrobium, Methylosarcina, and Methylocystis.

### BACKGROUND OF THE INVENTION

Pink Pigmented Facultative Methylotrophs (PPFMs) are alphaproteobacteria that have been shown to have beneficial symbiotic associations with a wide range of crop plants. Reported benefits from treatment of plants with PPFM bacteria include positive effects on nitrogen metabolism and seed germination, inhibition of fungal disease, reduction of plant damage from insects and/or nematodes, and stimulation of plant growth. The use of PPFM bacteria to improve plant growth, plant yield, seed germination, and plant nutritional qualities has been disclosed. Methanotrophic bacteria, or methanotrophs, are present in a wide variety of environments, and are able to grow on methane as their carbon source. Methanotrophs can be used in a variety of agricultural and industrial processes, including production of compounds of commercial interest, bioremediation of pollutants, and mitigation of methane gas.

To provide large quantities of PPFM and/or methanotroph inoculants for agricultural and/or industrial applications, growth in controlled bioreactors, or fermenters is desired. However, maintaining sterility during large scale fermentation can be problematic. Large scale fermentations can become contaminated with bacteria from equipment, media components, process water or other sources. Contamination, typically gram-positive bacterial strains, during the fermentation process can reduce final biomass, recovery efficiency and stability of formulations, resulting in reduced titers of PPFM and/or methanotroph bacteria, or even total loss of fermented bacteria in cases of high levels of contaminant growth.

Contaminating bacteria, predominantly lactic acid bacteria, have been a problem in yeast fermentations, typically of mash or molasses, for ethanol production for either fuel or brewing. Hop acids are commonly used in the brewing of beer where hop alpha acids contribute bitter flavor to the beer and have been shown to have antibiotic/bacteriostatic effects against gram-positive bacteria without affecting brewing yeast at the optimum beer brewing pH range of 5.2-5.6. The antibacterial effect of hop acids has been shown to decrease as pH increases from pH 4 to pH 7. U.S. Pat. No. 6,547,971 reports the use of alpha or beta hop acids in aqueous systems for inhibiting growth of and/or killing organisms, including gram-positive and gram-negative bacteria.

### SUMMARY OF THE INVENTION

Methods and compositions for growth of pink-pigmented facultative methylotrophs (PPFM) and/or methanotroph bacteria are provided herein, wherein hop acids are employed to inhibit growth of bacterial contaminants.

In one embodiment, a method disclosed herein comprises the steps of a) providing a sterile fermentation medium comprising nutrients suitable for growth of PPFM and/or methanotroph bacteria; b) adding hop acids to the sterile fermentation medium; c) adding an inoculum of PPFM and/or methanotroph cells to the fermentation medium to provide a fermentation broth; and d) maintaining the fermentation broth under conditions suitable for growth of the PPFM and/or methanotroph bacteria; whereby the growth of bacterial contaminants is inhibited. In one embodiment, the pH of the fermentation is greater than pH 6. In one embodiment, the hop acids are present at a concentration sufficient to inhibit growth of gram positive bacteria at pH 6 or higher. In one embodiment of the claimed methods, the hop acids comprise beta acids at a concentration of at least 7.5 ppm. In one embodiment the hop acids are a mixture of alpha and beta acids. In one embodiment the hop acids contain a higher percentage of beta acids than alpha acids. In one embodiment the hop acids are primarily beta acids. In one embodiment, hop beta acids are present in the fermentation at a concentration of at least about 7.5, 8, 8.5. 9. 9.5, 10, 12, 15, 20, 25, 30, 35, 40, 45, 50, 75, 100, 200, 300, 400, 500, 600, 700, 800, 900, or 1000 ppm or more. In one embodiment of the claimed methods, hop acids are added to the fermentation medium as a 10% dilution in methanol for ease of handling. In one embodiment of the present methods, the PPFM and/or methanotroph bacteria are grown in the fermentation medium to a titer of at least 10⁹ CFU per milliliter. In one embodiment, the methods disclosed herein further comprise harvesting the PPFM and/or methanotroph bacteria grown in the fermentation broth. In other embodiments, hop acids are employed in PPFM and/or methanotroph cultivation for manufacture of chemicals, food products, proteins and other products. Examples of products from PPFM and/or methanotroph cultivation include biochemicals, such as PHA, PHB, carotenoids, amino acids, terpenoids, polyketides, formate, and glyoxylate. In such embodiments, manufactured products can be harvested separately from the fermentation broth or may be harvested together with PPFM and/or methanotroph cells produced in the fermentation.

In one embodiment, a fermentation broth provided herein comprises nutrients to support growth of PPFM and/or methanotroph bacteria, a growing population of PPFM bacteria, and hop acids. In one embodiment, the population of PPFM bacteria in said fermentation broth is at a titer of at least 106 colony forming units (CFU) per milliliter. In one embodiment, beta hop acids are present at a concentration of at least 7.5 ppm. In one embodiment, the pH of the fermentation broth is greater than 6. In one embodiment, the hop acids are a mix of alpha and beta acids. In one embodiment, the fermentation broth contains a higher percentage of beta acids. In one embodiment, the hop acids in the fermentation are primarily beta acids. In one embodiment, hop beta acids are present in a fermentation at a concentration of at least about 7.5, 8, 8.5. 9. 9.5, 10, 12, 15, 20, 25, 30, 35, 40, 45, 50, 75, 100, 200, 300, 400, 500, 600, 700, 800, 900, or 1000 ppm or more. In one embodiment the PPFM and/or methanotroph population is at a titer of at least 10⁹ colony forming units per milliliter. In one embodiment, hop acids are present in compositions comprising harvested PPFM and/or methanotroph cells and provide additional benefits during storage, formulation and application of such compositions for further use.

### DETAILED DESCRIPTION

### Definitions

The term “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features or encompassing the items to which they refer while not excluding any additional unspecified features or unspecified items.

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “biological” refers to a component of a composition for treatment of plants or plant parts comprised of or derived from a microorganism. Biologicals include biocontrol agents, other beneficial microorganisms, microbial extracts, natural products, plant growth activators or plant defense agents. Non-limiting examples of biocontrol agents include bacteria, fungi, beneficial nematodes, and viruses. In certain compositions, a biological can comprise a mono-culture or co-culture of Methylobacterium, or a combination of Methylobacterium strains or isolates that have been separately cultured.

The term “PPFM” refers to a Pink Pigmented Facultative Methylotrophs, a type of gram-negative bacteria that are capable of growing on one carbon compounds, such as methanol, as the sole source of carbon and energy, but can also utilize more complex organic compounds, such as sugars, alcohols and organic acids. “PPFM” refers to pink pigmented facultative methyltrophs, bacteria in the genera “Methylobacterium” and Methylorubrum genus. Although defined as pink-pigmented for convenience, PPFM as used herein also encompasses non-pink pigmented Methylobacterium species, as well as colorless mutants of Methylobacterium or Methylorubrum isolates. For example, and not by way of limitation, PPFM refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus;

Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extorquens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense; Methylobacterium mesophilicum; Methylobacterium thiocyanatum (Methylorubrum thiocyanatum); Methylobacterium nodulans; Methylobacterium zatmanii (Methylorubrum zatmanii); Methylobacterium symbiota; or Methylobacterium organophilum.

As used herein nutrients suitable for growth of PPFM bacteria refers to nutrients used in a fermentation broth for production of PPFM bacteria. Such nutrients include fermentable sugars or alcohols as carbon source, including oligosaccharides and monosaccharides that can be used as a carbon source by a PPFM in a fermentation process. Carbon sources for use in the methods provided herein include, but are not limited to glycerol, fructose, methanol, ethanol, glutamic acid, sucrose, MSG, betaine, trimethylglycine, aspartic acid, and succinic acid.

As used herein, the term “methanotrophic bacteria” or “methanotroph” refers to genera and species of bacteria that are capable of using methane as their carbon source for growth. Methanotrophic bacteria include species in the genera Methyloacidimicrobium, Methyloacidiplilum, Methylobacter, Methylocaldum, Methylocapsa, Methylocella, Methylococcus, Methylocystis, Methyloferula, Methylogaea, Methyloglobus, Methylohalobius, Methylomagnum, Methylomarinum, Methylomicrobium, Methylomonas, Methyloparacoccus, Methyloperedens, Methyloprofundus, Methylosarcina, Methylosinus, Methylosoma, Methylosphaera, Methylothermus, and Methylovulum.

As used herein nutrients suitable for growth of methanotroph bacteria refers to nutrients used in a fermentation broth for production of methanotroph bacteria. Such nutrients include C1 substrates such as methane, methanol, formaldehyde, formic acid (formate), carbon monoxide, carbon dioxide, methylated amines, methylated thiols, or methyl halogens. In some embodiments, a fermentation broth may comprise a single C₁ substrate as the sole carbon source, or may comprise a mixture of two or more C₁ substrates to provide multiple carbon sources.

The term “contaminants” refers to the presence of microorganisms that are not intentionally introduced. Microorganisms, other than PPFM, that are present in the fermentation broth are considered contaminants.

The term “substantial contamination” refers to a level of bacterial contamination in a fermentation broth that would result in decreased yield of PPFM.

The term “fermentation medium” refers herein to a composition that supports the growth of PPFM bacteria. Fermentation medium may be used in any size including small scale cultures and large scale production fermentations.

The term “fermentation broth” refers to a composition comprising fermentation medium and a PPFM inoculum.

The term “hop acids” refers to a product of CO₂ extraction from hops, the female flowers of the hops plant Humulus lupulu. Extracted hop acids are composed of alpha acids (humulones) and beta acids (lupulones). Derivatives of hop acids can be produced, for example by boiling or chemical reduction, and such derivatives are also considered hop acids herein. Hop alpha acids include, but are not limited to, humulone, cohumulone, adhumulone, posthumulone and prehumulone. Hop beta acids include, but are not limited to, colupulone, lupulone, and adlupulone.

As used herein, the term “strain” shall include all isolates of such strain.

The term “starter culture” is a culture of PPFM cells that is used to inoculate a larger volume of fermentation medium to produce a fermentation broth.

### Fermentation

A useful antimicrobial agent for fermentations for selectively targeting contaminating bacteria, primarily gram positive bacteria, while not inhibiting the growth of the PPFM and/or methanotroph bacteria, is provided herein. Methods and fermentation broths comprising PPFM and/or methanotroph bacteria are disclosed, where such methods and fermentation broths include the use of hop acids to inhibit the growth of bacterial contaminants while allowing for the efficient growth of PPFM and/or methanotroph bacteria. Undesired, contaminating bacteria can be introduced in a fermentation process from biomaterial, process equipment, inoculation cultures, process water, air, or other sources. Controlling contamination in a fermentation allows the PPFM and/or methanotroph bacteria to grow and produce at a higher level than is attained in the presence of contaminating bacteria, and can increase yield of a desired fermentation product, prevent fermentation yield loss or prevent loss of an entire fermentation batch, thus providing a more efficient and economical fermentation process.

Hop acids are typically used in the brewing industry and known to perform better under low pH conditions, typically from a pH of about 5.2 to about 5.6. The antibacterial effect of hop acids has been shown to decrease as pH increases from pH 4 to pH 7. The examples provided herein demonstrate that not only do hop acids control gram positive contaminants at pH 6.0 and higher, but they also do not demonstrate antimicrobial or bacteriostatic effect on PPFM or methanotroph bacteria, as has been reported previously for various species of gram negative bacteria.

Hop acids are obtained from the female flower clusters of the hops plant Humulus lupulu, by carbon dioxide (CO₂) extraction. The extracted material may be further separated and/or chemically modified to produce hop acid preparations with varying proportions of alpha and beta hops acids, or modified forms of hop acids, such as isomerized forms of alpha acids. Various types of hop acids preparations are commercially available from manufacturers such as BetaTec and HopSteiner. In some embodiments, a hop acid product used in the present methods and compositions comprises predominantly beta hop acids.

HopSteiner Beta Bio 45% is a preparation that is 45% natural hop beta-acids. BetaTec IsoStab is an aqueous solution of isomerized alpha acids. BetaTec BetaStabXL is an aqueous formulation of primarily beta acids (8.5-9.5% w/w) emulsified with fatty acids. Of particular interest are the use of products containing a high proportion of beta hop acids, and use of such products to attain a beta acid concentration of about 7.5 ppm or higher in the fermentation broths and methods provided herein. Methods for extraction and purification of hop acids and enrichment for particular types of hop acids, including beta hop acids are known in the art. Concentrations of hop acids can vary in different hop varieties and varieties known to have a higher proportion of beta hop acids compared to alpha hop acids can be identified and provide a source of hop acids for use in the present methods and compositions.

In one embodiment provided herein, hop acids and a starter culture of PPFM and/or methanotroph cells are added to a sterile fermentation medium to produce a fermentation broth, which is maintained under conditions suitable for growth of the PPFM and/or methanotroph cells. In one embodiment, hop acids are added to a sterile fermentation media, followed by addition of PPFM and/or methanotroph cells to provide a fermentation broth. In other embodiments, hop acids and PPFM and/or methanotroph cells are added to the fermentation medium at the same time, or PPFM and/or methanotroph cells are added to the medium followed by the addition of hop acids. Thus, the present fermentation broth compositions comprise fermentation medium, hop acids, and a growing population of PPFM and/or methanotroph cells. Following inoculation, cells grow and form a growing population of PPFM and/or methanotroph cells in a fermentation broth.

The fermentation medium may be of any type that supports growth of PPFM and/or methanotroph bacteria. In some embodiments, the fermentation medium is prepared from readily available components, including, but not limited to, inorganic salts such as potassium phosphate, magnesium sulfate and the like, carbon sources such as glycerol, methanol, ethanol, glutamic acid, fructose, sucrose, MSG, betaine, trimethylglycine, aspartic acid, succinic acid and the like, and amino acid blends such as peptone, tryptone, and the like. Exemplary defined media that can be used for PPFM fermentation include, but are not limited to, ammonium mineral salts (AMS) medium (Whittenbury et al., 1970), Vogel-Bonner (VB) minimal culture medium (Vogel and Bonner, 1956), and LB broth (“Luria-Bertani Broth”). In some embodiments, the fermentation medium may contain solid substances that enhance growth of PPFM bacteria, such as particulate solids (U.S. Pat. No. 10,920,214). In some embodiments, the fermentation medium may be an emulsion, for example comprising a continuous aqueous phase containing nutrients for PPFM growth and a dispersed phase comprising a non-aqueous liquid that is immiscible or only partially miscible in the continuous phase (U.S. Pat. No. 10,287,544).

For example, disclosed is a method for inhibiting growth of bacterial contaminants in a culture medium comprising pink pigmented facultative methylotroph (PPFM) bacteria and/or methanotroph bacteria, wherein said method comprises: adding hop acids to a sterile fermentation medium comprising nutrients suitable for growth of PPFM and/or methanotroph bacteria; adding an inoculum of PPFM and/or methanotroph cells to the fermentation medium to provide a fermentation broth; and maintaining the fermentation broth under conditions suitable for growth of the PPFM and/or methanotroph bacteria; whereby the growth of bacterial contaminants is inhibited.

In some embodiments, PPFM and/or methanotroph bacteria are grown in a fermentation medium for production of an industrial product or compound, for example biodegradable plastics, such as PHA and PHB, glyoxylate and carotenoids, or for conversion of CO₂ to high value products, such as amino acids, terpenoids, polyketides, and formate. In some embodiments PPFM and/or methanotroph cells are harvested following fermentation and further processed to produce dried or liquid inoculant cultures for use in agriculture and/or methane mitigation methods.

In one embodiment the pH of a fermentation medium or broth provided herein is in the range of about pH 6.0 to about pH 8.0. In one embodiment, the pH of fermentation media and broth provided herein is in the range of about pH 6.5 to pH 7.5.

The amount of hop acids needed to control contamination in large scale fermentation will depend on various factors such as the source and amount of contamination, the media used in the fermentation, the proportion of alpha and beta hop acids in the hop acids product added, the concentration of PPFM and/or methanotroph cells following inoculation, and other fermentation conditions. One skilled in the art can adjust the amount of hop acids needed according to the observed fermentation conditions. In some embodiments, addition of concentrated hop acids which can be viscous products, is facilitated by dissolving the hop acid product in methanol to provide a 10% dilution.

The concentration of hop acids useful for inhibiting growth of bacterial contaminants in a PPFM and/or methanotroph fermentation broth can vary depending on the percentage of alpha and beta acids in the hop acid preparation. Examples provided herein demonstrate that hop acids were not effective in preventing growth of bacterial contaminants when added to complex media agar plates at a concentration of 10 parts per million of product, which resulted in a final hop acid concentration on the plates of from approximately 1 to 4.5 ppm hop acids depending on the concentration in a given product. Hop acid products that contain primarily alpha hop acids also failed to inhibit growth of gram positive contaminants when 100 or 1000 ppm of the hop acid product was added to plates, with hop acids present at final concentrations of from about 10-300 ppm. Hop acid products containing primarily beta hop acids are demonstrated herein to inhibit growth of all gram positive bacteria tested when added to media plates at 100 or 1000 ppm of the hop acid products, resulting in final beta hop acid concentrations of about 9-450 ppm. One product, Beta Bio 45, contained beta hop acids at a concentration of 45%, resulting in a concentration of 45 or 450 ppm in the fermentation medium when added to a concentration of 100 or 1000 ppm of the product. Another product, BetaStab XL contained 8.5 to 9.5% hop beta acids, resulting in a concentration in the fermentation medium of approximately 9 or 90 ppm when added to a concentration of about 100 or 1000 ppm of product. Thus, in the present compositions and methods, beta hop acids find use for inhibition of contamination of PPFM fermentation broths when provided at a concentration of greater than about 7.5 ppm. Of interest are methods and compositions for fermentation of PPFM and/or methanotroph bacteria wherein beta hop acids are provided at a concentration of at least about 7.5, 8, 8.5. 9. 9.5, 10, 12, 15, 20, 25, 30, 35, 40, 45, 50, 75, 100, 200, 300, 400, 500, 600, 700, 800, 900, or 1000 ppm or more (including any integer or fraction between) beta hop acids.

The PPFM and/or methanotroph inoculum may be any source of cells effective for starting a fermentation broth culture. Typically, cells are stored as frozen stocks and revived by growing a starter culture in a defined medium. The starter culture is added to the fermentation medium to produce a fermentation broth, or culture. In some embodiments, contamination in a starter culture can be controlled by addition of hop acids dissolved in methanol as described herein. In such instances, methanol can also act as an antimicrobial agent, and when the seed culture is used to inoculate a larger scale fermentation, it may not be necessary to add additional hop acids to the fermentation medium separately from the inoculum. Thus, in one embodiment, contamination is controlled in a fermentation by the inclusion of hop acids in a seed culture that is used to inoculate the fermentation medium.

Any strain of PPFM and/or methanotroph bacteria can be used in the methods and fermentation broths provided herein. In some embodiments the PPFM and/or methanotroph bacteria are useful for inoculation of plants to improve plant germination, nitrogen use efficiency, growth, root mass, and yield, and/or provide resistance to various organisms and plant pests, including bacteria, fungi, insects and nematodes. In some embodiments, the PPFM and/or methanotroph are mutants or engineered strains that produce increased levels of useful industrial compounds, nutrients or plant growth regulators. U.S. Pat. No. 8,153,118 discloses various Methylobacterium isolates that produce increased levels of vitamin B-12 and amino acids that can be used in the methods and compositions provided herein. Fermentation broths, fermentation broth products, and compositions that comprise one or more of the Methylobacterium such as Methylobacterium mutant B12-11 having accession number ATCC PTA-1561 that overproduces vitamin B-12, Methylobacterium rhodinum (ATCC #43282) that over-produces the amino acid threonine, Methylobacterium sp. (ATCC #21371) that over-produces the amino acid L-glutamic acid, Methylobacterium sp. (ATCC #21372) that over-produces the amino acid L-glutamic acid, Methylobacterium sp. (ATCC #21926) over-produces the amino acid L-lysine, Methylobacterium sp. (ATCC #21969) over-produces the amino acid L-glutamic acid, Methylobacterium sp. (ATCC #21927) over-produces the amino acids L-lysine, L-aspartic acid, L-alanine, L-valine, L-leucine, and L-arginine, and/or Methylobacterium sp. (ATCC #21438) that produces single-cell protein are also provided.

Non-limiting examples of Methylobacterium strains that can be used in methods provided herein are disclosed in Table 1. Other Methylobacterium strains useful in certain methods provided herein include variants of the Methylobacterium strains disclosed in Table 1. Also of use are various combinations of two or more strains or variants of Methylobacterium strains disclosed in Table 1 for treatment of plants or parts thereof.

TABLE 1
Methylobacterium sp. strain

| | | USDA ARS |
|---|---|---|
| Deposit Identifier | NLS ID | NRRL No.¹ |
| Methylobacterium sp. #1 | NLS0046 | NRRL B-50929 |
| Methylobacterium sp. #2 | NLS0020 | NRRL B-50930 |
| Methylobacterium sp. #3 | NLS0017 | NRRL B-50931 |
| Methylobacterium sp. #4 | NLS0042 | NRRL B-50932 |
| Methylobacterium sp. #5 | NLS0089 | NRRL B-50933 |
| Methylobacterium sp. #6 | NLS0068 | NRRL B-50934 |
| Methylobacterium sp. #7 | NLS0065 | NRRL B-50935 |
| Methylobacterium sp. #8 | NLS0069 | NRRL B-50936 |
| Methylobacterium sp. #9 | NLS0062 | NRRL B-50937 |
| Methylobacterium sp. #10 | NLS0064 | NRRL B-50938 |
| Methylobacterium sp. #11 | NLS0021 | NRRL B-50939 |
| Methylobacterium sp. #12 | NLS0066 | NRRL B-50940 |
| Methylobacterium sp. #13 | NLS0037 | NRRL B-50941 |
| Methylobacterium sp. #14 | NLS0038 | NRRL B-50942 |
| Methylobacterium sp. #15 | NLS0044 | NRRL B-67339 |
| Methylobacterium sp. #16 | NLS0109 | NRRL B-67340 |
| Methylobacterium sp. #17 | NLS0934 | NRRL B-67341 |
| Methylobacterium sp. #18 | NLS0648 | NRRL B-67741 |
| Methylobacterium sp. #19 | NLS0662 | NRRL B-67742 |
| Methylobacterium sp. #20 | NLS0807 | NRRL B-67743 |
| Methylobacterium sp. #21 | NLS0476 | NRRL B-67809 |
| Methylobacterium sp. #26 | NLS0610 | NRRL B-67892 |
| Methylobacterium sp. #22 | NLS0497 | NRRL B-67925 |
| Methylobacterium sp. #23 | NLS0693 | NRRL B-67926 |
| Methylobacterium sp. #25 | NLS1181 | NRRL B-67927 |
| Methylobacterium sp. #24 | NLS1179 | NRRL B-67929 |
| Methylobacterium sp. #28 | NLS0433 | NRRL B-68032 |
| Methylobacterium sp. #29 | NLS1026 | NRRL B-68033 |
| Methylobacterium sp. #30 | NLS1084 | NRRL B-68034 |
| Methylobacterium sp. #31 | NLS0668 | NRRL-B-68064 |
| Methylobacterium sp #32 | NLS0687 | NRRL B-68065 |
| Methylobacterium sp #33 | NLS0722 | NRRL B-68066 |
| Methylobacterium sp #34 | NLS0834 | NRRL B-68067 |
| Methylobacterium sp #35 | NLS0940 | NRRL B-68068 |
| Methylobacterium sp #36 | NLS0947 | NRRL B-68069 |
| Methylobacterium sp #37 | NLS0737 | NRRL-B-68074 |
| Methylobacterium sp #38 | NLS0770 | NRRL-B-68075 |
| Methylobacterium sp. #39 | NLS5278 | NRRL-B-68186 |
| Methylobacterium sp. #40 | NLS5334 | NRRL-B-68187 |
| Methylobacterium sp. #41 | NLS5480 | NRRL-B-68188 |
| Methylobacterium sp. #42 | NLS5549 | NRRL-B-68189 |
| Methylobacterium sp #43 | NLS0665 | NRRL-B-68194 |
| Methylobacterium sp #44 | NLS0729 | NRRL-B-68195 |
| Methylobacterium sp #45 | NLS0672 | NRRL-B-68196 |
| Methylobacterium sp #46 | NLS0754 | NRRL-B-68197 |
| Methylobacterium sp #47 | NLS0591 | NRRL-B-68215 |
| Methylobacterium sp #48 | NLS0439 | NRRL-B-68216 |
| Methylobacterium sp #49 | NLS1310 | NRRL-B-68217 |
| Methylobacterium sp #50 | NLS1312 | NRRL-B-68218 |
| Methylobacterium sp #51 | NLS0049 | NRRL-B-68236 |
| Methylobacterium sp #52 | NLS0612 | NRRL-B-68237 |
| Methylobacterium sp #53 | NLS0706 | NRRL-B-68238 |
| Methylobacterium sp #54 | NLS0725 | NRRL-B-68239 |
| Methylomicrobium sp. 55 | NLS1501 | NRRL B-68261 |
| Methylosarcina sp. 56 | NLS1504 | NRRL B-68281 |
| Methylocystis sp. 57 | NLS1505 | NRRL B-68282 |
| Methylocystis sp. 58 | NLS1506 | NRRL B-68283 |
| Methylocystis sp. 59 | NLS1508 | NRRL B-68262 |
| Methylocystis sp. 60 | NLS1509 | NRRL B-68284 |
| Methylocystis sp. 61 | NLS1511 | NRRL B-68285 |
| Methylocystis sp. 62 | NLS1512 | NRRL B-68286 |
| Methylorubrum sp. #63 | NLS7725 | NRRL B-68260 |

¹Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR §1.808(b), all restrictions imposed by the depositor on the availability to the public of the deposited material will be irrevocably removed upon the granting of any patent from this patent application.

In some embodiments provided herein, the inoculated culture medium, or fermentation broth, is incubated under conditions suitable for growth of PPFM and/or methanotroph bacteria. Typically, temperature is between about 30° C. and about 37° C., and pH is about 6.5 to about 7.5. Antifoam agents, such as dimethopolysiloxane, may be added to the medium as needed to minimize foaming. In some embodiments, PPFM fermentations for production of PPFM inoculants are conducted as a fed-batch fermentation and run for 1-7 days, and PPFM concentrations of 1×10⁸ to about 1×10¹¹ CFU per milliliter are obtained. In some embodiments, PPFM fermentations for production of PPFM inoculants are conducted as a fed-batch fermentation and run for 1-4 days, 1-3 days or 1-2 days. Fermentation for production of methanotroph bacteria may be conducted as a batch or continuous culture process. For growth of methanotroph bacteria a C1 carbon source such as methane or methanol will be employed. Exemplary defined media for growth of methanotroph bacteria are described, for example, in WO2021071966 and include Higgins minimal nitrate salts medium (NSM), MM-W1 medium, master mix feed (MMF), medium MMF1.1, medium MMS1.0, and AMS medium. Media and conditions for culture of PPFM and/or methanotroph bacteria can be optimized depending on the strain or strains of bacteria selected for growth.

Control of contaminating bacteria may be assessed by overlay plating of fermentation broth samples. In some embodiments PPFM and/or methanotroph cells are harvested following fermentation and further processed to produce dried or liquid inoculant cultures for use in agriculture, bioremediation and/or methane mitigation methods. Dried preparations can be prepared, for example, by spray drying, freeze drying, air drying, fluid bed drying, electrospray drying, or other drying methods.

For commercial production fermentation cultures, a variety of culture methodologies may be applied. For example, large-scale production may use both batch and continuous culture methodologies. A classical batch culturing method is a closed system where the composition of the medium is set at the beginning of the culture and not subjected to artificial alterations during the culturing process. Thus, at the beginning of the culturing process the medium is inoculated with the desired organism and growth or metabolic activity is permitted to occur. Typically, however, a “batch” culture is batch with respect to the addition of carbon source, and adjustments are made to control factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of ethanol.

A variation on the standard batch system is the Fed-Batch system. Fed-Batch culture processes are also suitable for the present methods and compositions, and comprise a typical batch system with the exception that the substrate is added in increments as the culture progresses. Measurement of the actual substrate concentration in Fed-Batch systems is difficult and is therefore estimated on the basis of the changes of measurable factors such as pH and the partial pressure of waste gases such as CO₂. Batch and Fed-Batch culturing methods are common and well known in the art and examples may be found in Biotechnology: A Textbook of Industrial Microbiology, Crueger, Crueger, and Brock, Second Edition (1989) Sinauer Associates, Inc., Sunderland, M A, or Deshpande, Mukund V., Appl. Biochem. Biotechnol., 36, 227, (1992)

The present methods and compositions may also be used in a continuous culture process. Continuous cultures are open systems where culture medium is added continuously to a bioreactor and an equal amount of conditioned medium is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials as is known to one skilled in the art.

Uses of PPFM and/or Methanotroph Fermentation Products

PPFM and/or methanotroph bacteria prepared using the media and methods provided herein may find use in agriculture. Microbial inoculant compositions are provided to a plant or a plant part, to soil where a plant is grown, to a nutrient solution in which a plant is grown, for example in hydroponic growing systems, to soil where a plant part such as a seed is deposited, in aeroponic applications, such as in a root mist, or any combinations thereof. Treatments or applications can include, but are not limited to, spraying, coating, partially coating, immersing, and/or imbibing the plant or plant parts with the compositions provided herein. In certain embodiments, a seed, a leaf, a vegetative cutting, a fruit, a stem, a root, a tuber, or a coleoptile can be immersed, dipped in, and/or imbibed with a liquid, semi-liquid, emulsion, or slurry of a composition provided herein. Plants and plant parts coated or partially coated with the microbial inoculant compositions are also provided herein.

Plant parts for treatment with PPFM and/or methanotroph bacteria are selected from the group consisting of a leaf, a stem, a fruit, a vegetative cutting, a flower, a root, a seedling, a tuber, or a seed. A PPFM and/or methanotroph inoculant is applied to a plant or plant part (e.g., a seed) at about 1×10², 1×10³, 1×10⁴, or 1×10⁵ to about 1×10⁷, 1×10⁸, 1×10⁹, or 1×10¹⁰ CFUs of PPFM and/or methanotroph bacteria per plant or plant part (e.g., a seed).

Plants which can be treated with PPFM and/or methanotroph compositions include corn, soybean, Brassica sp. (e.g., B. napus, B. rapa, B. juncea), alfalfa, rice, rye, wheat, barley, oats, sorghum, millet (e.g., pearl millet (Pennisetum glaucum)), proso millet (Panicum miliaceum), foxtail millet (Setaria italica), finger millet (Eleusine coracana), sunflower, safflower, tobacco, potato, peanuts, cotton, species in the genus Cannabis (including, but not limited to, Cannabis sativa and industrial hemp varieties), sweet potato (Ipomoea batatus), cassava, coffee, coconut, pineapple, citrus trees, cocoa, tea, date palm, banana, apple, pear, grape, berry plants (including, but not limited to blackberry, raspberry, strawberry or blueberry plants), avocado, fig, guava, kiwi, mango, olive, papaya, cashew, macadamia, almond, sugar beets, sugarcane, tomatoes, peppers, lettuce, leafy greens (including, but not limited to, spinach, kale, escarole microgreens, collard greens, cabbage, beet greens, watercress, romaine lettuce, swiss chard, arugula, endive, bok choy and turnip greens), green beans, lima beans, peas, lentils, cucurbits (including, but not limited to cucumber, cantaloupe, melons, squash, pumpkin, and zucchini). Other leafy green plants that are grown for production and harvest of microgreens and/or herbs, can also be treated with PPFM including, but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, carrot, fennel, beans, peas, chickpeas, and lentils. In other embodiments, treated plants include ornamentals (including, but not limited to, azalea, hydrangea, hibiscus, roses, tulips, daffodils, petunias, carnation, poinsettia, Pennisetum, and chrysanthemum), conifers (including, but not limited to pines such as loblolly pine, slash pine, ponderosa pine, lodge pole pine, and Monterey pine; Douglas-fir; Western hemlock; Sitka spruce; redwood; true firs such as silver fir and balsam fir; and cedars such as Western red cedar and Alaska yellow-cedar) and turfgrass (including, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass).

Microbial inoculant compositions for agricultural applications are provided which comprise PPFM and/or methanotroph bacteria at a titer of greater than about 5×10⁷ colony-forming units per milliliter, at a titer of greater than about 1×10⁸ colony-forming units per milliliter, at a titer of greater than about 5×10⁸ colony-forming units per milliliter, at a titer of greater than about 1×10⁹ colony-forming units per milliliter, at a titer of greater than about 1×10¹⁰ colony-forming units per milliliter, at a titer of at least about 3×10¹⁰ colony-forming units per milliliter. In certain embodiments, microbial inoculant compositions provided herein comprise PPFM and/or methanotroph bacteria at a titer of at least about 5×10⁷, 1×10⁸, or 5×10⁸ colony-forming units per milliliter to at least about 4×10¹⁰ colony-forming units per milliliter, at least about 5×10⁸ colony-forming units per milliliter to at least about 4×10¹⁰ colony-forming units per milliliter, or at least about 5×10⁸ colony-forming units per milliliter to at least about 6×10¹⁰ colony-forming units per milliliter. In certain embodiments, microbial inoculant compositions provided herein comprise PPFM and/or methanotroph bacteria at a titer of at least about 1×10⁹ colony-forming units per milliliter to at least about 3×10¹⁰ colony-forming units per milliliter, at least about 1×10⁹ colony-forming units per milliliter to at least about 4×10¹⁰ colony-forming units per milliliter, or at least about 1×10⁹ colony-forming units per milliliter to at least about 6×10¹⁰ colony-forming units per milliliter. In certain embodiments, microbial inoculant compositions provided herein comprise PPFM and/or methanotroph bacteria at a titer of at least about 1×10¹⁰ colony-forming units per milliliter to at least about 3×10¹⁰ colony-forming units per milliliter, at least about 1×10¹⁰ colony-forming units per milliliter to at least about 4×10¹⁰ colony-forming units per milliliter, or at least about 1×10¹⁰ colony-forming units per milliliter to at least about 6×10¹⁰ colony-forming units per milliliter. In certain embodiments, microbial inoculant compositions provided herein comprise PPFM and/or methanotroph bacteria at a titer of, at least about 3×10¹⁰ colony-forming units per milliliter to at least about 4×10¹⁰ colony-forming units per milliliter, or at least about 3×10¹⁰ colony-forming units per milliliter to at least about 6×10¹⁰ colony-forming units per milliliter. In any of the aforementioned compositions, the compositions can be essentially free of contaminating microorganisms, can comprise PPFM and/or methanotroph bacteria that are adhered to and/or associated with materials that the PPFM and/or methanotroph bacteria are not adhered to and/or associated with in nature, or any combination thereof.

In certain embodiments of any of the foregoing compositions and methods, dried preparations or microbial inoculant compositions with PPFM and/or methanotroph bacteria at a titer of greater than about 5×10⁷, 1×10⁸, or 5×10⁸ colony-forming units per gram, at a titer of greater than about 1×10⁹ colony-forming units per gram, at a titer of greater than about 1×10¹⁰ colony-forming units per gram, at a titer of at least about 3×10¹⁰ colony-forming units per gram are provided. In certain embodiments, microbial inoculant compositions provided herein comprise PPFM and/or methanotroph bacteria at a titer of at least about 5×10⁷, 1×10⁸, or 5×10⁸ colony-forming units per gram to at least about 3×10¹⁰ colony-forming units per gram, at least about 5×10⁷, 1×10⁸, or 5×10⁸ colony-forming units per gram to at least about 4×10¹⁰ colony-forming units per gram, or at least about 5×10⁷, 1×10⁸, or 5×10⁸ colony-forming units per gram to at least about 6×10¹⁰ colony-forming units per gram. In certain embodiments, microbial inoculant compositions provided herein can comprise PPFM and/or methanotroph bacteria at a titer of at least about 1×10⁹ colony-forming units per gram to at least about 3×10¹⁰ colony-forming units per gram, at least about 1×10⁹ colony-forming units per gram to at least about 4×10¹⁰ colony-forming units per gram, or at least about 1×10⁹ colony-forming units per gram to at least about 6×10¹⁰ colony-forming units per gram. In certain embodiments, microbial inoculant compositions provided herein will comprise PPFM and/or methanotroph bacteria at a titer of at least about 1×10¹⁰ colony-forming units per gram to at least about 3×10¹⁰ colony-forming units per gram, at least about 1×10¹⁰ colony-forming units per gram to at least about 4×10¹⁰ colony-forming units per gram, or at least about 1×10¹⁰ colony-forming units per gram to at least about 6×10¹⁰ colony-forming units per gram. In certain embodiments, microbial inoculant compositions provided herein will comprise PPFM and/or methanotroph bacteria at a titer of, at least about 3×10¹⁰ colony-forming units per gram to at least about 4×10¹⁰ colony-forming units per gram, or at least about 3×10¹⁰ colony-forming units per gram to at least about 6×10¹⁰, 1×10¹¹, 1×10¹², 1×10¹³, or 5×10¹³ colony-forming units per gram. In any of the aforementioned microbial inoculant or compositions, the composition can comprise PPFM and/or methanotroph bacteria that is adhered to a solid substance. In any of the aforementioned microbial inoculant compositions, the compositions can be essentially free of contaminating microorganisms, can comprise PPFM and/or methanotroph bacteria that are adhered to and/or associated with materials that the PPFM and/or methanotroph bacteria are not adhered to and/or associated with in nature, or any combination thereof.

In some embodiments, the compositions or methods disclosed herein may comprise one or more PPFM and/or methanotroph bacteria isolates and an additional active ingredient, which may be, for example, a pesticide, a non-biological plant or microbial stimulant, or a second biological. In certain embodiments, the pesticide can be an insecticide, a fungicide, an herbicide, a nematicide, or other biocide. The second biological could be a strain that improves yield or controls an insect, pest, fungi, weed, or nematode. In some embodiments, a second biological is a second PPFM and/or second methanotroph strain.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, and thiodicarb.

Non-limiting examples of useful fungicides include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. Non-limiting examples of other biocides include isothiazolinones, for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT); 2-Bromo-2-nitro-propane-1,3-diol (Bronopol), 5-bromo-5-nitro-1,3-dioxane (Bronidox), Tris(hydroxymethyl) nitromethane, 2,2-Dibromo-3-nitrilopropionamide (DBNPA), and alkyl dimethyl benzyl ammonium chlorides.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins. Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, the composition or method disclosed herein may comprise a PPFM and/or methanotroph strain and an additional active ingredient selected from the group consisting of clothianidin, ipconazole, imidacloprid, metalaxyl, mefenoxam, tioxazafen, azoxystrobin, thiomethoxam, fluopyram, prothioconazole, pyraclostrobin, and sedaxane.

In some embodiments, compositions or methods disclosed herein may comprise an additional active ingredient which may be a plant or microbial biostimulant. In some embodiments, an additional active ingredient may be a second biological. The second biological could be a biocontrol agent, other beneficial microorganisms, microbial extracts, plant extracts (botanicals), and other natural products, including for example, protein hydrolysates and other nitrogen containing compounds, plant growth activators or stimulants or a plant defense agent. Examples of useful additional components include yeast or seaweed or extracts and powders thereof, humic and fulvic acids, amino acids, peptides, chitosan and other biopolymers, plant hormones, such as auxins and cytokinins or derivatives thereof, trace elements and nucleic acids. In some embodiments, compositions provided herein contain combinations of any of these or related compounds. Non-limiting examples of biocontrol agents include bacteria, fungi, beneficial nematodes, and viruses.

In certain embodiments, the second biological can be a bacterium of the genus Actinomycetes, Agrobacterium, Arthrobacter, Alcaligenes, Aureobacterium, Azobacter, Azorhizobium, Azospirillum, Azotobacter, Beijerinckia, Bacillus, Brevibacillus, Burkholderia, Chromobacterium, Clostridium, Clavibacter, Comomonas, Corynebacterium, Curtobacterium, Enterobacter, Flavobacterium, Gluconacetobacter, Gluconobacter, Herbaspirillum, Hydrogenophage, Klebsiella, Luteibacter, Lysinibacillus, Mesorhizobium, Methylobacterium, Microbacterium, Ochrobactrum, Paenibacillus, Pantoea, Pasteuria, Phingobacterium, Photorhabdus, Phyllobacterium, Pseudomonas, Rhizobium, Rhodococcus, Bradyrhizobium, Serratia, Sinorhizobium, Sphingomonas, Streptomyces, Stenotrophomonas, Variovorax, Xanthomonas and Xenorhadbus. In particular embodiments, the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments the second biological can be a fungus of the genus Acremonium, Alternaria, Ampelomyces, Aspergillus, Aureobasidium, Beauveria, Botryosphaeria, Cladosporium, Cochliobolus, Colletotrichum, Coniothyrium, Embellisia, Epicoccum, Fusarium, Gigaspora, Gliocladium, Glomus, Laccaria, Metarhisium, Muscodor, Nigrospora, Paecilonyces, Paraglomus, Penicillium, Phoma, Pisolithus, Podospora, Rhizopogon, Scleroderma, Trichoderma, Typhula, Ulocladium, and Verticilium. In particular embodiments, the fungus is Beauveria bassiana, Coniothyrium minitans, Gliocladium vixens, Muscodor albus, Paecilomyces lilacinus, or Trichoderma polysporum.

In further embodiments the second biological can be plant growth activators or plant defense agents including, but not limited to harpin, Reynoutria sachalinensis, jasmonate, lipochitooligosaccharides, and isoflavones.

In further embodiments, the second biological can include, but is not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or wild-type isolates that are available as pure cultures. In certain embodiments, it is anticipated that the second biological can be provided in the composition in the form of a spore.

In further embodiments of any of the foregoing compositions or methods, additional components may include agricultural excipients and/or agricultural adjuvants. Agriculturally acceptable adjuvants used in the compositions include, but are not limited to, components that enhance product efficacy and/or products that enhance ease of product application. Adjuvants that enhance product efficacy can include various wetters/spreaders that promote adhesion to and spreading of the composition on plant parts, stickers that promote adhesion to the plant part, penetrants that can promote contact of the active agent with interior tissues, extenders that increase the half-life of the active agent by inhibiting environmental degradation, and humectants that increase the density or drying time of sprayed compositions. Wetters/spreaders used in the compositions can include, but are not limited to, non-ionic surfactants, including, but not limited to alkylpolyglucosides (APGs), polysorbate/sorbitan based surfactants (e.g. Tween/Span), fatty alcohol ethoxylates, fatty acid ethoxylates, alkylphenol ethoxylates, fatty glycerol esters, block copolymers (e.g., Poloxamers); anionic surfactants; cationic surfactants; amphoteric surfactants, such as phospholipids (including but not limited to phosphatidylserine, phosphatidylethanolamine, phosphatidylcholine, and sphingomyelins) cocamidoporpyl based surfactants (including but not limited to betaines and sultaines); organo-silicate surfactant wetting agents (including but not limited to trisiloxane, and polyether siloxanec); and/or acidified surfactants. Stickers used in the compositions can include, but are not limited to, film forming substances, such as calcium alginate or other mineral alginates, gelatin, drying oils (such as tung oil and linseed oil), and polyvinyl alcohols/acrylates; latex-based substances, terpene/pinolene, and pyrrolidone-based substances. Penetrants can include mineral oil, vegetable oil, esterified vegetable oil, organo-silicate surfactants, and acidified surfactants. Extenders used in the compositions can include, but are not limited to, ammonium sulphate, or menthene-based substances. Humectants used in the compositions can include, but are not limited to, glycerol, propylene glycol, and diethyl glycol. Adjuvants that improve ease of product application include, but are not limited to, dispersants, acidifying/buffering agents, anti-foaming/de-foaming agents, compatibility agents, drift-reducing agents, dyes, and water conditioners. Dispersants that improve ease of use and handling for homogenous mixing and application of product include, for example, acrylate polymers or copolymers, lignosulfonates, (alkyl) naphthalene sulfonate. Anti-foaming/de-foaming agents used in the compositions can include, but are not limited to, dimethopolysiloxane. Compatibility agents used in the compositions can include, but are not limited to, ammonium sulphate. Drift-reducing agents used in the compositions can include, but are not limited to, polyacrylamides, and polysaccharides. Water conditioners used in the compositions can include, but are not limited to, ammonium sulphate.

In certain embodiments, PPFM compositions used to treat a plant or plant seed can contain agriculturally acceptable excipients and/or adjuvants. Such excipients include, but are not limited to, woodflours, clays, activated carbon, diatomaceous earth, fine-grain inorganic solids, calcium carbonate and the like. Clays and inorganic solids that can be used with the compositions provided herein include, but are not limited to, calcium bentonite, kaolin, china clay, talc, graphite, perlite, mica, vermiculite, silicas, quartz powder, montmorillonite and mixtures thereof. In certain embodiments where plant seeds are treated with PPFM compositions, the compositions further comprise one or more lubricants to ensure smooth flow and separation (singulation) of seeds in the seeding mechanism, for example a planter box. Lubricants for use in such compositions include talc, graphite, polyethylene wax-based powders (such as Fluency Agent), protein powders, for example soybean protein powders, or a combination of protein powders and a lipid, for example lecithin or a vegetable oil. Combinations of such lubricants may also be used, including, for example blends of clay and talc. Lubricants can be applied to seeds simultaneously with application of inoculant or may be mixed with the inoculant prior to application of the compositions to the seeds. Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, alginates, dextrins, malto-dextrins, polysaccharides, fats, oils, proteins, karaya gum, jaguar gum, tragacanth gum, polysaccharide gums, mucilage, gum arables, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylimide polymers and copolymers, polyhydroxyethyl acrylate, methylacrylimide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Various surfactants, dispersants, anticaking-agents, foam-control agents, and dyes disclosed herein and in U.S. Pat. No. 8,181,388 can be adapted for use in compositions provided herein.

### DEPOSIT INFORMATION

Samples of the following Methylobacterium sp. strains have been deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylobacterium sp. NRRL B-50929, NRRL B-50930, NRRL B-50931, NRRL B-50932, NRRL B-50933, NRRL B-50934, NRRL B-50935, NRRL B-50936, NRRL B-50937, NRRL B-50938, NRRL B-50939, NRRL B-50940, NRRL B-50941 and NRRL B-50942 were deposited with NRRL on Mar. 12, 2014. Methylobacterium sp. NRRL B-67339, NRRL B-67340, and NRRL B-67341 were deposited with NRRL on Nov. 18, 2016. Methylobacterium sp. NRRL B-67741, NRRL B-67742, NRRL B-67743 were deposited with NRRL on Dec. 20, 2018. Methylobacterium sp. NRRL B-67809 was deposited with NRRL on Jun. 28, 2019. Methylobacterium sp. NRRL B-67892 was deposited with NRRL on Nov. 26, 2019. Methylobacterium sp. NRRL B-67925, NRRL B-67926 and NRRL B-67927 were deposited with NRRL on Feb. 21, 2020. Methylobacterium sp. NRRL B-67929 was deposited with NRRL on Mar. 3, 2020. Methylobacterium sp. NRRL B-68032, NRRL B-68033 and NRRL B-68034 were deposited with NRRL on May 20, 2021. Methylobacterium sp. NRRL B-68064, NRRL B-68065, NRRL B-68066, NRRL B-68067, NRRL B-68068, and NRRL B-68069 were deposited with NRRL on Sep. 9, 2021. Methylobacterium sp. NRRL B-68074 and NRRL B-68075 were deposited with NRRL on Oct. 6, 2021. Methylobacterium sp. NRRL B-68186, NRRL B-68187, NRRL B-68188 and NRRL B-68189 were deposited with NRRL on Aug. 3, 2022. Methylobacterium sp. NRRL B-68194, NRRL B-68195, NRRL B-68196, and NRRL B-68197 were deposited with NRRL on Aug. 30, 2022. Methylobacterium sp. NRRL B-68215, NRRL B-68216, NRRL B-68217, and NRRL B-68218 were deposited with NRRL on Nov. 2, 2022. Methylobacterium sp. NRRL B-68236, NRRL B-68237, NRRL B-68238, and NRRL B-68239 were deposited with NRRL on Nov. 23, 2022. Methylomicrobium sp. NRRL B-68261 and Methylosarcina sp. NRRL B-68262 were deposited with NRRL on Feb. 14, 2023. Methylorubrum sp. NRRL B-68260 was deposited with NRRL on Mar. 9, 2023. Methylosarcina sp. NRRL B-68281, and Methylocystis sp. NRRL B-68282, NRRL B-68283, NRRL B-68284, NRRL B-68285, and NRRL B-68286 were deposited with NRRL on Jun. 7, 2023.

Subject to 37 CFR § 1.808 (b), all restrictions imposed by the depositor on the availability to the public of the deposited material will be irrevocably removed upon the granting of any patent from this patent application.

Having described the embodiments in detail, it will be apparent that modifications and variations are possible without departing from the scope defined in the appended claims.

### EXAMPLES

### Example 1: PPFM and Methanotroph Tolerance to Hop Acids

A complex media plate (glutamate/Phytopeptone) pH 6.8 with an overlay of PPFM bacterial was prepared by adding 100 μL of several different PPFM strains to 4 ml of 0.7% agar of the same media. Hop acids tested were Hopsteiner Beta Bio 45% (containing 45% beta acids), BetaTec IsoStab (an aqueous solution containing 28.0-32.0% isomerized alpha acids), BetaTec BetaStabXL (containing 8.5 to 9.5% hop beta acids), and BetaTec FermaHop Pro (containing 9.5-10.5% hop acids consisting primarily of alpha acids). 10 μL of a solution of 10, 100 or 1000 ppm of each hop acids product was either added to acro discs and placed on the overlay or deposited in a small circle directly on the overlay. Final concentrations of hop acids added to plates for the tested products are shown in Table 2 below.

TABLE 2

| | | | 10 ppm | | 100 ppm | | 1000 ppm | |
|---|---|---|---|---|---|---|---|---|
| Product | Hop acids % | Hop acids type | dilution | | dilution | | dilution | |
| Hopsteiner Beta Bio 45% | 45% | beta acids | 4.5 | ppm | 45 | ppm | 450 | ppm |
| BetaTec BetaStabXL | 8.5-9.5% | beta acids | 0.9 | ppm | 9.0 | ppm | 90 | ppm |
| BetaTec IsoStab | 28.0-32.0% | iso-alpha acids. | 3 | ppm | 30 | ppm | 300 | ppm |
| BetaTec FermaHop Pro | 9.5-10.5 | hop bitter acids | 1 | ppm | 10 | ppm | 100 | ppm |

Several different species of PPFM were tested and no growth inhibition of any strains was seen with addition of 10, 100 or 1000 ppm hop acids.

To demonstrate tolerance of methanotroph bacteria to hop acids, 100 ul aliquots of cultures of Methylomicrobium, Methylosarcina, and one or more Methylocystis strains disclosed in Table 1 are spread plated onto carbon-free mineral salts media (such as Nitrate Mineral Salts (NMS), pH 6.8. 10 μL of a solution of 10, 100 or 1000 ppm of hop acids product will be either added to acro discs and placed on the plates or deposited in a small circle directly on the plates. The plates will be incubated under methane gas and monitored for the development of a bacterial lawn of growth indicative of tolerance of the methanotroph strains to hop acids.

### Example 2: Hop Acids Effects on Gram Positive Bacteria

Overlay plates were prepared as described in Example 1 with various strains of gram positive bacteria in the overlay. Gram positive bacteria tested were Bacillus weidmannii, Lysinibacillus fusiformis, or Rhodococcus corynebacterioides. Hop acid products were tested at 10, 100 and 1000 ppm yielding final hop acid concentrations as shown in Table 2 above. None of the tested hop acid products demonstrated any zone of inhibition indicative of growth inhibition when applied at 10 ppm to final hop acid concentrations of from about 1-4.5 ppm. When applied at 100 or 1000 ppm to final concentrations of from 9-450 ppm, the high beta acid hop extract products resulted in zones of growth inhibition for all of the gram positive species tested. Hop acid products containing primarily alpha acids did not inhibit growth of any of the gram positive species tested when applied at 100 or 1000 ppm to final hop acid concentrations of 10-300 ppm.

### Example 3: Hops Acids Effects on Contaminants in Fermentation Media

Two PPFM strains were grown with and without added Lysinibacillus fusiformis (Lf) or Bacillus weidmannii (Bw) contaminants, and with or without 45 ppm beta hop acids. 100 ppm Beta Bio 45% was added post media sterilization to a minimal fermentation media pH 6.8 containing fructose (PPFM strain 1 experiment) or glycerol (PPFM strain 2 experiment) as the carbon source, resulting in a final concentration of beta hop acids of 45 ppm. Initial PPFM concentrations were approximately 10⁷ CFU per ml with contaminant bacteria added to a titer of 10⁵ per ml. PPFM and contaminant bacterial counts were determined at inoculation (Time 0) and at 27, and 53 hours post inoculation. Contaminant counts in media containing hop acids were also determined at 77 hours post inoculation.

TABLE 3
PPFM Strain 1 in Minimal Media plus Fructose

| | | | | PPFM1 + | | LPPFM1 + |
|---|---|---|---|---|---|---|
| | PPFM1 | | PPFM1 + | Bw | PPFM1 + | Lf |
| Time | No hop | PPFM1 + | Bw + | No hop | Lf + | No hop |
| (h) | acids | hop acids | hop acids | acids | hop acids | acids |
| Mtroph Counts | | | | | | |
| 0 | 3.4 × 10⁷ | 3.2 × 10⁷ | | 3.1 × 10⁷ | 3.2 × 10⁷ | 3.1 × 10⁷ |
| 27 | 3.0 × 10⁹ | 3.0 × 10⁹ | 2.0 × 10⁹ | | 8.0 × 10⁸ | |
| 53 | 1.1 × 10¹⁰ | 1.1 × 10¹⁰ | 1.5 × 10¹⁰ | 1.4 × 10⁹ | 1.7 × 10⁹ | 6.9 × 10⁹ |
| 77 | | | | | | |
| Contaminant Counts | | | | | | |
| 0 | N/A | N/A | 0 | 0 | 10 | 30 |
| 27 | N/A | N/A | 0 | 6.0 × 10⁶ | 2.0 × 10³ | 8.0 × 10⁸ |
| 53 | N/A | N/A | 0 | 1.5 × 10⁸ | 0 | 8.0 × 10⁸ |
| 77 | | | 2.0 × 10² | | 2.0 × 10² | |

TABLE 4
PPFM Strain 2 in Minimal Media plus Glycerol

| | | | | PPFM2 + | | LPPFM2 + |
|---|---|---|---|---|---|---|
| | PPFM2 | | PPFM2 + | Bw | PPFM2 + | Lf |
| Time | No hop | PPFM2 + | Bw + | No hop | Lf + | No hop |
| (h) | acids | hop acids | hop acids | acids | hop acids | acids |
| Mtroph Counts | | | | | | |
| 0 | 2.7 × 10⁷ | 2.3 × 10⁷ | 2.8 × 10⁷ | 2.4 × 10⁷ | 2.7 × 10⁷ | 2.6 × 10⁷ |
| 27 | 1.5 × 10⁹ | 7.5 × 10⁸ | 8.0 × 10⁸ | | 1.5 × 10⁹ | |
| 53 | 5.0 × 10⁹ | 4.9 × 10⁹ | 7.5 × 10⁹ | 1.1 × 10¹⁰ | 7.4 × 10⁹ | 2.6 × 10⁹ |
| 77 | | | | | | |
| Contaminant Counts | | | | | | |
| 0 | N/A | N/A | 0 | 0 | 10 | 20 |
| 27 | N/A | N/A | 0 | 2.0 × 10⁷ | 0 | 5.0 × 10⁸ |
| 53 | N/A | N/A | 0 | 5.5 × 10⁸ | 1.0 × 10³ | 9.0 × 10⁸ |
| 77 | | | 2.0 × 10⁴ | | 6.0 × 10³ | |

These tests showed no increase in the contaminant bacteria counts even after 77 hours of growth. The counts decreased to 1×10⁴ CFU per ml or lower but were still detectable. The hop acids appear to be bacteriostatic, but not bactericidal to the gram positive contaminants tested.

PPFM strains were present at the beginning of the experiments at a concentration of at least 10⁷ CFU per ml following inoculation and increased in number to 10⁹ or 10¹⁰ CFU per ml after 53 hours of growth. Six different PPFM strains tested in this way showed no growth inhibition in fermentation media containing hop acids.

What is claimed:

1. A method for inhibiting growth of bacterial contaminants in a culture medium comprising pink pigmented facultative methylotroph (PPFM) bacteria and/or methanotroph bacteria, wherein said method comprises: (a) providing a sterile fermentation medium comprising nutrients suitable for growth of PPFM and/or methanotroph bacteria; (b) adding hop acids to the sterile fermentation medium; (c) adding an inoculum of PPFM and/or methanotroph cells to the fermentation medium to provide a fermentation broth; and (d) maintaining the fermentation broth under conditions suitable for growth of the PPFM and/or methanotroph bacteria; whereby the growth of bacterial contaminants is inhibited.

2. The method of claim 1 wherein the pH of said fermentation broth is greater than pH 6.

3. The method of claim 1 or 2 wherein said bacterial contaminants are gram positive bacteria.

4. The method of any one of claims 1 to 3 wherein said hop acids are present at a concentration sufficient to inhibit growth of gram positive bacteria at pH 6 or higher.

5. The method of any one of claims 1 to 4 wherein said hop acids comprise beta acids at a concentration of at least 7.5 ppm.

6. The method of any one of claims 1 to 5 wherein said hop acids comprise beta acids at a concentration of at least 45 ppm.

7. The method of any one of claims 1 to 6 wherein said hop acids comprise beta acids at a concentration of about 100 ppm.

8. The method of any one of claims 1 to 7 wherein said hop acids are a mixture of alpha and beta acids.

9. The method of any one of claims 1 to 8 wherein said hop acids contain a higher percentage of beta acids than alpha acids.

10. The method of claim 9 wherein said hop acids are primarily beta acids.

11. The method of any one of claims 1 to 10 wherein said hop acids are added to said fermentation medium as a 10% dilution in methanol.

12. The method of any one of claims 1 to 11 wherein said PPFM and/or methanotroph bacteria are grown to a titer of at least 10⁹ CFU per milliliter.

13. The method of any one of claims 1 to 12 wherein said method further comprises harvesting the PPFM and/or methanotroph bacteria grown in the fermentation broth.

14. The method of any one of claims 1 to 13 wherein said method further comprises harvesting one or more chemicals or proteins produced in said fermentation.

15. The method of any one of claims 1 to 14 wherein said culture medium comprises PPFM bacteria.

16. The method of any one of claims 1 to 14 wherein said culture medium comprises methanotroph bacteria.

17. The method of any one of claims 1 to 14 wherein said culture medium comprises PPFM and methanotroph bacteria.

18. A fermentation broth comprising nutrients, a growing population of pink pigmented facultative methylotroph (PPFM) bacteria and/or methanotroph bacteria, and hop acids, wherein said population of PPFM and/or methanotroph bacteria is at a titer of at least 1×10⁷ CFU per milliliter, and said hop acids comprise beta acids at a concentration of at least 7.5 ppm.

19. The fermentation broth of claim 18, wherein the pH of said fermentation broth is greater than pH 6.

20. The fermentation broth of claim 18 or 19, wherein said hop acids are a mixture of alpha and beta acids.

21. The fermentation broth of any one of claims 18 to 20, wherein said hop acids contain a higher percentage of beta acids than alpha acids.

22. The fermentation broth of any one of claims 18 to 21, wherein said hop acids are primarily beta acids.

23. The fermentation broth of any one of claims 18 to 22, wherein said PPFM and/or methanotroph population is at a titer of at least 1×10⁹ CFU per milliliter.

24. The fermentation broth of any one of claims 18 to 23, wherein said hop beta acids are at a concentration of at least 45 ppm.

25. The fermentation broth of any one of claims 18 to 24 wherein said hop acids comprise beta acids at a concentration of about 100 ppm.

26. The fermentation broth of any one of claims 18 to 25 wherein said fermentation broth comprises PPFM.

27. The fermentation broth of any one of claims 18 to 25 wherein said fermentation broth comprises methanotroph bacteria.

28. The fermentation broth of any one of claims 18 to 25 wherein said fermentation broth comprises PPFM and methanotroph bacteria.
